(19) United States
(12) Patent Application Publication
(10) Pub. No.: US 2019/0341126 A1
(43) Pub. Date: Nov. 7, 2019

(54) SYSTEM FOR THE QUANTIFICATION OF SYSTEM-WIDE DYNAMICS IN COMPLEX NETWORKS

(71) Applicant: Prime Genomics, Inc., Santa Cruz, CA (US)

(72) Inventor: Sandy C. Shaw, San Francisco, CA (US)

(21) Appl. No.: 16/298,557

(22) Filed: Mar. 11, 2019

Related U.S. Application Data
(63) Continuation of application No. 14/991,502, filed on Jan. 8, 2016, now abandoned.
Application No. 14/991,502 is a continuation-in-part of application No. 13/135,466, filed on Jul. 6, 2011, now abandoned.
(60) Provisional application No. 61/362,676, filed on Jul. 8, 2010.

(51) Int. Cl. G16B 25/00; G16B 20/00

(52) U.S. Cl. CPC G16B 25/00; G16B 20/00

(57) ABSTRACT

A device, method and system are provided for diagnosing a disease using a gene expression reader to analyze biological samples and output gene expression values to calculate a scaling factor using a computer by counting a number of link counts Cₙ for groups of an individual genes' expression values at different times at a threshold value C or for groups of genes' expression values at a single time at the threshold value C, calculating an average number Cₐᵥₑ of the link counts Cₙ, calculating a largest number M of the Cₙ, iteratively applying a relation Cₐᵥₑ=M/log(M) for different threshold values C, comparing data of the Cₐᵥₑ values versus M/log(M), and calculating a fitting to the compared data to output the scaling factor a. The scaling factor a is compared with other scaling factors a′ in a database to output a report of estimates for a degree of health.

### CROSS-REFERENCE TO RELATED APPLICATIONS

This application is a continuation of U.S. application Ser. No. 14/991,502, filed Jan. 8, 2016, which is a continuation-in-part of U.S. application Ser. No. 13/135,466, filed Jul. 6, 2011, which claims the benefit of U.S. Provisional Application Ser. No. 61/362,676, filed Jul. 8, 2010, all of which are incorporated herein by reference in their entirety.

### FIELD OF THE INVENTION

The present invention relates to diagnosing disease. More particularly, the invention relates to analyzing biological samples for gene expression values to determine a degree of health of the biological sample.

### BACKGROUND OF THE INVENTION

A large, complex network of interacting components is difficult to describe as a whole dynamic system. In genetics research, scientists examining large numbers of genes, or genetic networks, often focus on identifying one gene or a group of genes that appears to be important to a particular outcome or pathology. What is needed are a low cost and efficient device, method and system for analyzing the interconnections between genes and genetic networks on a large-scale to output a report of a degree of health in a patient.

### SUMMARY OF THE INVENTION

To address the needs in the art, a method of diagnosing a disease is provided, according to one embodiment of the invention, that includes using a gene expression reader analyzing at least one biological sample, where the gene expression reader includes a probe interfacing the at least one biological sample, where the probe includes a fragment of nucleic acid having a specific sequence of bases that uniquely match a region of interest of a gene in a genome of the biological sample, where the probe interrogates a specific gene or a region within the specific gene of the biological sample, where the probe quantifies the expression level of the gene in the biological sample and outputs gene expression values from at least two genes based on the analyzing at least one biological sample, and outputting gene expression values from at least two genes based on analyzing the biological samples, calculating a scaling factor a for the biological samples using an appropriately programmed computer, where the scaling factor a is calculated from the gene expression values by counting a number of link counts Cₙ for groups of an individual genes' expression values at different times at a threshold value C, or for groups of genes' expression values at a single time at the threshold value C, calculating an average number Cₐᵥₑ of the link counts Cₙ calculating a largest number M of the Cₙ, where the M includes the largest of the number of link counts Cₙ for a given threshold value C for all the gene expression value groups, iteratively applying a relation Cₐᵥₑ=M/log(M) for different threshold values C, comparing data of the Cₐᵥₑ values versus M/log(M), and calculating a fitting to the compared data to output the scaling factor a, where the scaling factor a is the slope of the fitting. The method further includes comparing values of the scaling factor a for the biological samples with other scaling factors a′ in a database from analyzed biological samples using the appropriately programmed computer, and outputting a report using the appropriately programmed computer, where the report includes estimates of the at least one biological sample for a degree of health.

According to one aspect of the current method embodiment, the at least one biological sample can include saliva, urine, other body fluids, synovial fluid, breast ductal fluid, blood and blood components, tissue, tumors, bone marrow, stem cells, induced pluripotent cells, cell lines, plant material, or other organic material.

In another aspect of the current method embodiment, the gene expression reader includes at least two gene probes.

In a further aspect of the current method embodiment, the number of link counts Cₙ includes a number of link counts for each of N expression value groups, where each expression value group includes a sequence of gene expression values n₁, n₂, . . . n_{T}, at a threshold value C between the expression value group and the sequence of gene expression values n₁, n₂, . . . n_{T} for the other N−1 gene expression value groups.

According to another aspect of the current method embodiment, the scaling factor a is calculated by iteratively applying Cₐᵥₑ=M/log(M) for different threshold values C, using the appropriately programmed computer, and comparing Cₐᵥₑ values versus M/log(M), and calculating a linear fitting of the comparison to get the scaling factor a.

In yet another aspect of the current method embodiment, comparing values of a further includes comparing byproducts of the scaling factor a, comparing healthy samples against disease samples, or comparing an unknown sample with a database of values from samples with a known condition.

According to another aspect of the current method embodiment, the threshold value C is in a range between 0 and 1.

In another embodiment of the invention, a system for diagnosing disease is provided that includes a gene expression reader for analyzing at least one biological sample, where the gene expression reader includes a probe interfacing the at least one biological sample, where the probe includes a fragment of nucleic acid having a specific sequence of bases that uniquely match a region of interest of a gene in a genome of the biological sample, where the probe interrogates a specific gene or a region within the specific gene of the biological sample, where the probe quantifies the expression level of the gene in the biological sample and outputs gene expression values from at least two genes based on the analyzing at least one biological sample, and outputting gene expression values of at least two genes, a computer server for receiving from the gene expression reader the gene expression values and for managing and communicating patient information to a user, and a computer program hosted on the computer server, where the computer program analyzes the gene expression values and outputs a report, where the report includes estimates of the at least one biological sample for a degree of health, where the estimate includes comparing a scaling factor a for the at least one biological sample with other scaling factors a′ in a database from previously analyzed biological samples, where the scaling factor a is calculated from the gene expression values using the computer program by counting a number of link counts Cₙ for groups of an individual genes' expression values at a different times at a threshold value C or for groups of genes' expression values at a single time at the threshold value C, calculating an average number Cₐᵥₑ of the link counts Cₙ, calculating a largest number M of the Cₙ, where the M includes the largest of the number of link counts Cₙ for a given threshold value C for all the gene expression value groups, iteratively applying a relation Cₐᵥₑ=M/log(M) for different threshold values C, comparing the Cₐᵥₑ data values versus M/log(M) data, and applying a fitting to the compared data to output the scaling factor a, where the scaling factor a is the slope of the fitting.

According to one aspect of the current system embodiment, the at least one biological sample can include saliva, urine, other body fluids, synovial fluid, breast ductal fluid, blood and blood components, tissue, tumors, bone marrow, stem cells, induced pluripotent cells, cell lines, plant material, or organic material.

In another aspect of the current system embodiment, the gene expression reader includes at least two gene probes.

In a further aspect of the current system embodiment, the number of link counts Cₙ includes a number of link counts for each of N expression value groups, where each expression value group includes a sequence of gene expression values n₁, n₂, . . . n_{T}, at a threshold value C between the expression value group and the sequence of gene expression values n₁, n₂, . . . n_{T} for the other N−1 gene expression value groups.

According to another aspect of the current system embodiment, the a scaling factor a is calculated by iteratively applying Cₐᵥₑ=M/log(M) for different threshold values C, using the appropriately programmed computer, and comparing Cₐᵥₑ values versus M/log(M) and calculating a linear fitting of the comparison to get the scaling factor a.

In yet another aspect of the current system embodiment, comparing values of a further includes comparing byproducts of the scaling factor a, comparing healthy samples against disease samples, or comparing an unknown sample with a database of values from samples with a known condition.

In a further aspect of the current system embodiment, the threshold value C is in a range between 0 and 1.

In another embodiment, the invention includes lab-on-a-chip device having a substrate for holding a biological sample receptacle, a gene expression reader and a microprocessor, where biological sample receptacle includes a sample input to the gene expression reader, where the gene expression reader outputs gene expression values of at least two genes based on analyzed the at least one biological sample, where the microprocessor includes a computer program for analyzing gene expressions in the at least one biological sample, where the computer program compiles the gene expression values, counts a number of link counts Cₙ for groups of an individual genes' expression values at different times at a threshold value C or for groups of genes' expression values at a single time at the threshold value C, calculates an average number Cₐᵥₑ of the link counts Cₙ, calculates a largest number M of the Cₙ, where the M includes the largest of the number of link counts Cₙ for a given the threshold value C for all the gene expression value groups, iteratively applies a relation Cₐᵥₑ=M/log(M) for different threshold values C, compares data of the Cₐᵥₑ values versus M/log(M) data, calculates a fitting to the compared data to output the scaling factor a, where the scaling factor a is the slope of the fitting, compares values of the scaling factor a for the at least one biological sample with other stored scaling factors a′ from analyzed biological samples, and outputs a report, where the report includes estimates of the at least one biological sample for a degree of health.

According to one aspect of the current device embodiment, the at least one biological sample can include saliva, urine, other body fluids, synovial fluid, breast ductal fluid, blood and blood components, tissue, tumors, bone marrow, stem cells, induced pluripotent cells, cell lines, plant material, or organic material.

In another aspect of the current device embodiment, the gene expression reader includes at least two gene probes.

In a further aspect of the current device embodiment, the number of link counts Cₙ includes a number of link counts for each of N expression value groups, where each expression value group includes a sequence of gene expression values n₁, n₂, . . . n_{T}, at a threshold value C between the expression value group and the sequence of gene expression values n₁, n₂, . . . n_{T} for the other N−1 gene expression value groups.

According to one aspect of the current device embodiment, the a scaling factor a is calculated by iteratively applying the Cₐᵥₑ=M/log(M) for different threshold values C, using the appropriately programmed computer, and comparing Cₐᵥₑ values versus M/log(M) and calculating a linear fitting the comparison to get the scaling factor a.

In a further aspect of the current device embodiment, comparing values of a further includes comparing byproducts of the scaling factor a, comparing healthy samples against disease samples, or comparing an unknown sample with a database of values from samples with a known condition.

In yet aspect of the current device embodiment, the threshold value C is in a range between 0 and 1.

### BRIEF DESCRIPTION OF THE DRAWINGS

FIG. 1 shows a flow diagram of a method of one embodiment of the current invention.

FIG. 2 shows a graphical image of the process used by a computer program to calculate the scaling factor, according to one embodiment of the current invention.

FIGS. 3A-3B show (3A) a diagram of a system of one embodiment of the current invention, and (3B) a diagram of the probe, according to one embodiment of the invention.

FIG. 4 shows a schematic drawing of a device of one embodiment of the current invention.

### DETAILED DESCRIPTION

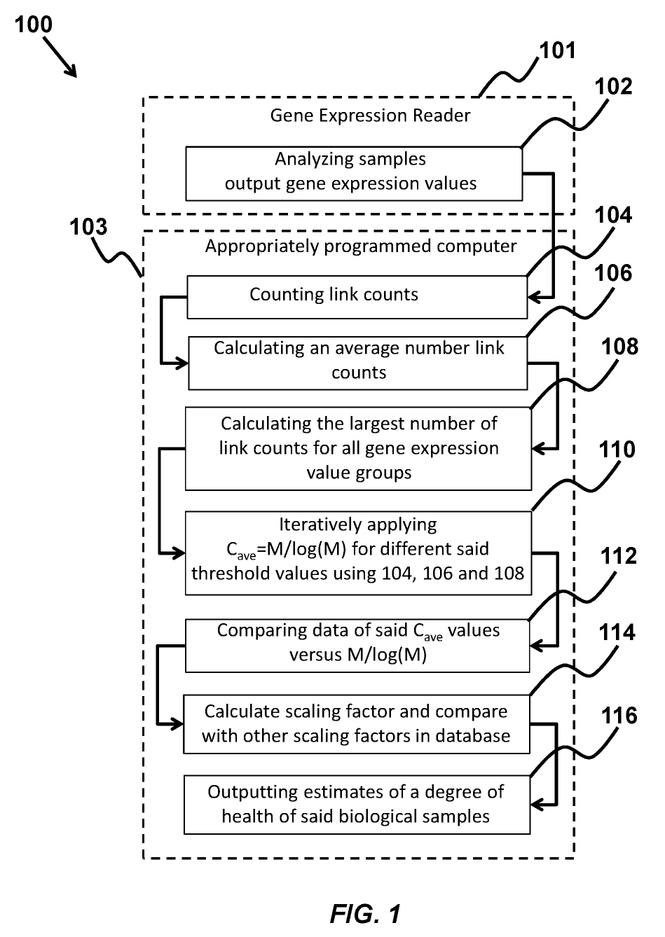

To address the needs in the art, a method of diagnosing a disease is provided, according to one embodiment of the invention. FIG. 1 shows a flow diagram of a method 100 of one embodiment of the invention, that includes a gene expression reader 101 analyzing at least one biological sample and outputting gene expression values 102 from at least two genes based on analyzing the at least one biological sample and use this to calculate a scaling factor a for the biological sample using an appropriately programmed computer 103, where the scaling factor a is calculated from the gene expression values by counting a number of link counts Cₙ 104 for groups of an individual genes' expression values at different times at a threshold value C or for groups of genes' expression values at a single time at the threshold value C, calculating an average number Cₐᵥₑ 106 of the link counts Cₙ, calculating a largest number M of the Cₙ 108, where the M includes the largest of the number of link counts Cₙ for a given threshold value C for all the gene expression value groups, iteratively applying a relation Cₐᵥₑ=M/log(M) for different threshold values C 110, comparing data of the Cₐᵥₑ values versus M/log(M) 112, and calculating a fitting to the compared data to output the scaling factor a, where the scaling factor a is the slope of the fitting and comparing values of the scaling factor a for the at least one biological sample with other scaling factors a′ 114 in a database from analyzed biological samples using the appropriately programmed computer, and outputting a report 116 using the appropriately programmed computer, where the report includes estimates of the at least one biological sample for a degree of health. In one aspect of the current embodiment, the gene expression reader includes at least two gene probes.

According to one embodiment, the invention enables the identification of sets of networks that are quantifiably different between two sample sets. The identification of these network nodes (genes) leads directly to the design of specific probes for these genes that allow for the interrogation of these specific genes and then quantification of the expression level of the gene from the sample, and then reading them for expression values in a gene expression reader. Specifically, the probe includes a short fragment of nucleic acid that has a specific sequence of bases that uniquely match that region of interest from that gene in the genome. In one aspect, the probes can be 15-20 nucleotides long. They match those unique regions in the genome by finding and binding to that specific gene that contains that complementary sequence of nucleotides. This match ideally only occurs once in the genome. According to the invention, the specific probes implemented are based on the specific sequences required to identify and match that gene or gene region in the genome, where the invention is identifying the genes by using the above algorithm. The identified genes lead to the design of specific probes for those genes that unambiguously allow for the interrogation of those genes then quantify the gene for level of gene expression. Gene expression levels are then compared between the at least two genes for at least one sample.

According to one embodiment of the method 100, the invention uses gene expression values, for example from a microarray or genechip, for N expression value groups that can include a large number, if not all, the genes in a genome for a given organism, for example. In one embodiment, N does not need to contain all available expression value groups of the microarray data, only a large subset of the microarray data.

In one embodiment of the method 100, the gene expression values n_{T} can be read from the microarray at multiple time intervals T. The dataset for quantification will include N groups of gene expression values n_{T} of the form:

n₁,n₂, . . . n_{T}

Where n is the gene expression value of of one of N genes taken at T intervals.

For the sequence of gene expression values nⱼ in the gene expression value group Nᵢ, the absolute value is taken of a correlation between the gene expression value group Nᵢ and every other gene expression value group (the other N−1 groups).

The total number of other gene expression value groups with a correlation above a threshold value C is called Cₙ and represents the number of links connecting this gene expression value group to all other gene expression value groups in the dataset with a value of C or greater. The largest of the Cₙ for a given C for all N gene expression value groups is then taken and called M. The average of all the Cₙ for a given C is also taken and called C_{avg}. According to one embodiment of the invention, for different values of C, the values of M and C_{avg} form the relation:

C_{avg}=(M/log(M))^{a}

To find the value of the scaling factor a, the method above is repeated by iteratively applying a relation Cₐᵥₑ=M/log(M) for different threshold values C, comparing the Cₐᵥₑ data values versus M/log(M) data, and applying a fitting to the compared data to output the scaling factor a, where the scaling factor a is the slope of the fitting. According to the current embodiment, the threshold value C is in a range between 0 and 1.

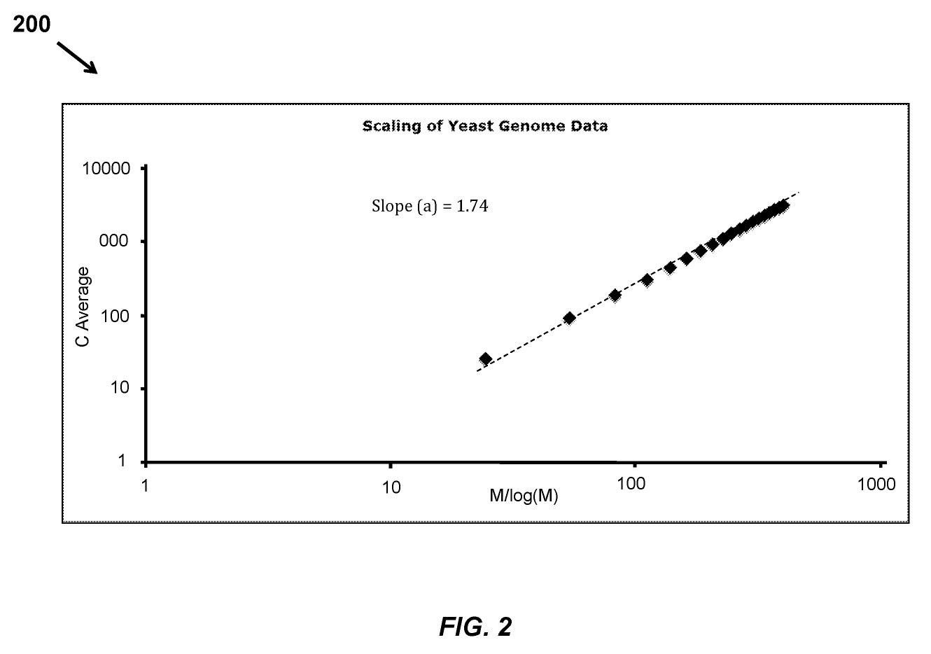

In one embodiment of the method 100, shown in FIG. 2 is an exemplary graphical scaling factor representation 200, where the number of values of cutoff value C is nineteen, C is the absolute value of the correlation, for example a Pearson correlation, and C ranges from 0.95 to 0.05 at decreasing values of 0.05 for each point. The slope of the line fitted to a log-log plot of the data is then measured. In this case a is shown to be ˜1.74. In FIG. 2, the correlation values measured are between time series of six gene expression values (T=6) taken at seven-minute intervals for 3360 genes (N=3360) in yeast (S. cerevisiae). Although 3360 genes are used in this example, the genes used in other examples can be any number, but are generally in the thousands. In one embodiment, it is possible to apply this method to groups of gene expression values measured at a single time rather than individual gene's expression values at different times. In other words, the correlation values are between N groups made up of gene expression values from T genes taken at a single time.

In one example of this embodiment, given gene expression values for 5 different genes at a single time labeled 1-5, three gene expression value groups (N=3) can be made containing three gene expression values each (T=3). For example, the gene expression values from genes 1-3, 2-4, 3-5. The invention calculates the absolute values of the Pearson correlation between each group, and the other two (N−1=2). Assume that 4 of the correlation values calculated are >0.95. Then Cₐᵥₑ for C=0.95 and N=3=4/3=1.33. Further, assume that the largest number of absolute Pearson correlation values >0.95 for any single gene expression value group is 2. Then M for C=0.95 would be 2.

The essence of both the single-time groups and the time series (time groups) approach is that in each case correlation values are taken between one group and all the other groups. Then it is calculated how many correlation values are greater that the threshold C. The largest number for any single group is M. The total number for all groups divided by the number of groups (N) gives Cₐᵥₑ. Though these are two different ways to calculate scaling factors a that could be different values, according to one aspect of the invention, the only requirement is that either method used to generate a must be consistent when comparing values of a between biological samples.

According to one aspect of the method 100, the at least one biological sample can include saliva, urine, other body fluids, synovial fluid, breast ductal fluid, blood and blood components, tissue, tumors, bone marrow, stem cells, induced pluripotent cells, cell lines, plant material, or other organic material.

In another aspect of the method 100, comparing values of a further includes comparing byproducts of the scaling factor a, comparing healthy samples against disease samples, or comparing an unknown sample with a database of values from samples with a known condition.

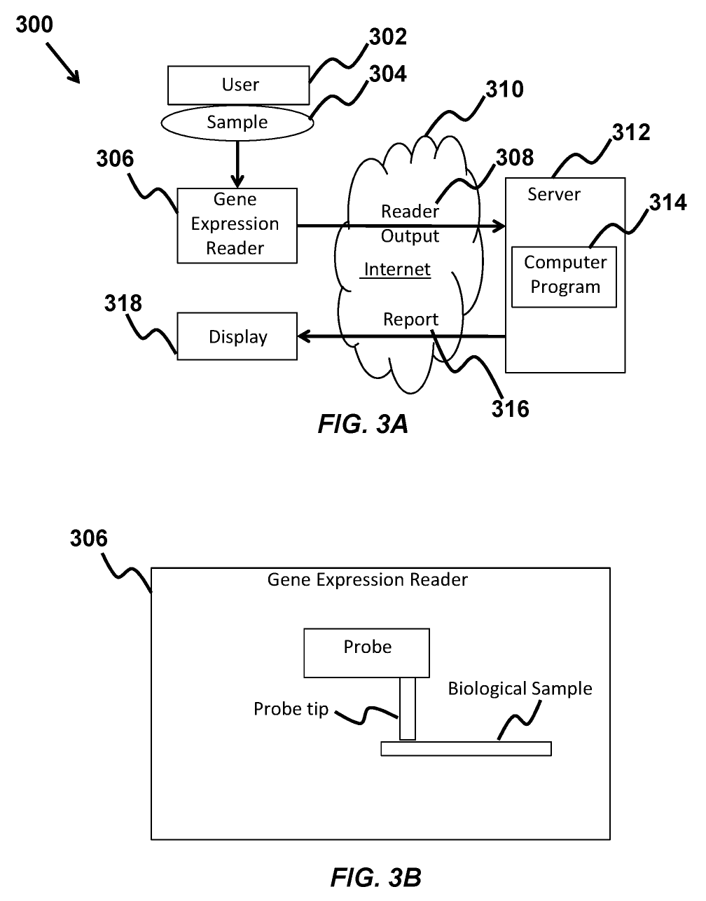

In another embodiment of the invention, FIG. 3A shows a system for diagnosing disease 300 that includes a user 302 having a biological sample 304 to input to a gene expression reader 306 for analyzing at least one biological sample 304 and outputting 310 gene expression values of at least two genes, and communicating 310 the gene expression values, for example using the internet, to a computer server 312 for receiving from the gene expression reader 306 the gene expression values and for managing and communicating patient information, where the patient information is then provided to the user 302. A computer program 314 is hosted on the computer server 312 and analyzes the gene expression values to then output a report 316 that can be viewed on a display 318 that includes estimates of the at least one biological sample for a degree of health. According to the current embodiment, the estimate includes comparing a scaling factor a for the at least one biological sample with other scaling factors a′ in a database from previously analyzed biological samples, where the scaling factor a is calculated from the gene expression values using the computer program 314 by counting a number of link counts Cₙ for groups of an individual genes' expression values at a different times at a threshold value C or for groups of genes' expression values at a single time at the threshold value C, calculating an average number Cₐᵥₑ of the link counts Cₙ, calculating a largest number M of the Cₙ, where the M includes the largest of the number of link counts Cₙ for a given threshold value C for all the gene expression value groups, iteratively applying a relation Cₐᵥₑ=M/log(M) for different threshold values C, comparing the Cₐᵥₑ data values versus M/log(M) data, and applying a fitting to the compared data to output the scaling factor a, where the scaling factor a is the slope of the fitting. FIG. 3B shows a diagram of the probe interrogating a biological sample.

According to one embodiment of the system 300, the at least one biological sample can include saliva, urine, other body fluids, synovial fluid, breast ductal fluid, blood and blood components, tissue, tumors, bone marrow, stem cells, induced pluripotent cells, cell lines, plant material, or organic material.

In another aspect of the system 300, the gene expression reader includes at least two gene probes.

In a further aspect of the system 300, the number of link counts Cₙ includes a number of link counts for each of N expression value groups, where each expression value group includes a sequence of gene expression values n₁, n₂, . . . n_{T}, at a threshold value C between the expression value group and the sequence of gene expression values n₁, n₂, . . . n_{T} for the other N−1 gene expression value groups.

According to another aspect of the system 300, the a scaling factor a is calculated by iteratively applying Cₐᵥₑ=M/log(M) for different threshold values C, using the appropriately programmed computer, and comparing Cₐᵥₑ values versus M/log(M) and calculating a linear fitting of the comparison to get the scaling factor a.

In yet another aspect of the system 300, comparing values of a further includes comparing byproducts of the scaling factor a, comparing healthy samples against disease samples, or comparing an unknown sample with a database of values from samples with a known condition.

In a further aspect of the system 300, the threshold value C is in a range between 0 and 1.

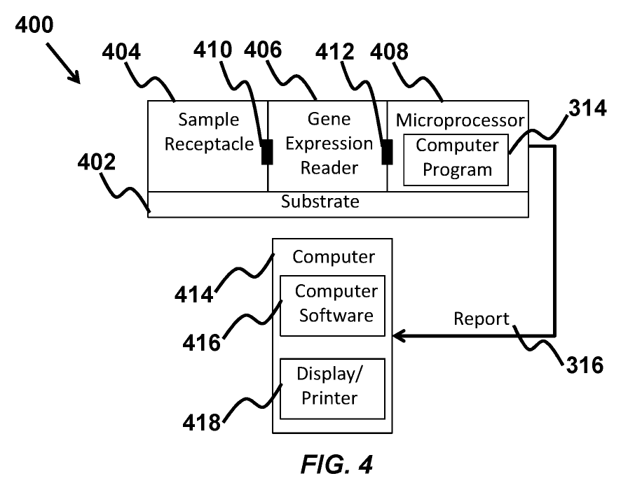

FIG. 4 shows another embodiment of the invention that includes lab-on-a-chip device 400 having a substrate 402 for holding a biological sample receptacle 404, a gene expression reader 406 and a microprocessor 408, where biological sample receptacle 404 includes a sample input 410 to the gene expression reader, where the gene expression reader outputs 412 gene expression values of at least two genes based on analyzed the at least one biological sample, where the microprocessor 408 includes a computer program 314 for analyzing gene expressions in the biological sample 304 input by the user 302 to the sample receptacle 404. The computer program 314 compiles the gene expression values, counts a number of link counts Cₙ for groups of an individual genes' expression values at different times at a threshold value C or for groups of genes' expression values at a single time at the threshold value C, calculates an average number Cₐᵥₑ of the link counts Cₙ, calculates a largest number M of the Cₙ, where the M includes the largest of the number of link counts Cₙ for a given the threshold value C for all the gene expression value groups, iteratively applies a relation Cₐᵥₑ=M/log(M) for different threshold values C, compares data of the Cₐᵥₑ values versus M/log(M) data, calculates a fitting to the compared data to output the scaling factor a, where the scaling factor a is the slope of the fitting, compares values of the scaling factor a for the at least one biological sample with other stored scaling factors a′ from analyzed biological samples, and outputs a report 316, where the report 316 includes estimates of the at least one biological sample for a degree of health. The report can be communicated to a computer 414 having computer software 416 and a display or printer 418. Further, it is understood that the substrate 402 can be any suitable platform, host or housing and that the computer 414 can be separate or integrated with the substrate 402.

According to one aspect of the device 400, the at least one biological sample can include saliva, urine, other body fluids, synovial fluid, breast ductal fluid, blood and blood components, tissue, tumors, bone marrow, stem cells, induced pluripotent cells, cell lines, plant material, or organic material.

In another aspect of the device 400, the gene expression reader includes at least two gene probes.

In a further aspect of the device 400, the number of link counts Cₙ includes a number of link counts for each of N expression value groups, where each expression value group includes a sequence of gene expression values n₁, n₂, . . . n_{T}, at a threshold value C between the expression value group and the sequence of gene expression values n₁, n₂, . . . n_{T} for the other N−1 gene expression value groups.

According to one aspect of the device 400, the a scaling factor a is calculated by iteratively applying the Cₐᵥₑ=M/log(M) for different threshold values C, using the appropriately programmed computer, and comparing Cₐᵥₑ values versus M/log(M) and calculating a linear fitting the comparison to get the scaling factor a.

In a further aspect of the device 400, comparing values of a further includes comparing byproducts of the scaling factor a, comparing healthy samples against disease samples, or comparing an unknown sample with a database of values from samples with a known condition.

In yet aspect of the device 400, the threshold value C is in a range between 0 and 1.

The present invention has now been described in accordance with several exemplary embodiments, which are intended to be illustrative in all aspects, rather than restrictive. Thus, the present invention is capable of many variations in detailed implementation, which may be derived from the description contained herein by a person of ordinary skill in the art. For example, other complex interconnected networks where a single network component or node in the network can have the degree to which is it switched “on” quantified in a way similar to single gene expression values in a genetic network. Examples could include: numbers characterizing the total energy that each single protein in a protein-protein interaction network acquires from binding with other proteins in the network, other biochemical networks where the interaction between single components and other components can be similarly quantified for each component, numbers reflecting the flow of information to/from each single node in a communication or computer network, and numbers reflecting the flow of traffic through individual intersections in a city traffic network or between individual hubs in a transportation network.

All such variations are considered to be within the scope and spirit of the present invention as defined by the following claims and their legal equivalents.

1. A method of diagnosing a disease, comprising:
a. using a gene expression reader to analyze at least one biological sample, wherein said gene expression reader comprises a probe interfacing said at least one biological sample, wherein said probe comprises a fragment of nucleic acid having a specific sequence of bases that uniquely match a region of interest of a gene in a genome of said biological sample, wherein said probe interrogates a specific gene or a region within said specific gene of said biological sample, wherein said probe quantifies the expression level of said gene in said biological sample and outputs gene expression values from at least two genes based on said analyzing said at least one biological sample;
b. calculating a scaling factor a for said at least one biological sample using an appropriately programmed computer, wherein said scaling factor a is calculated from said gene expression values comprising:
i. counting a number of link counts Cₙ for groups of individual genes' expression values at different times at a threshold value C or for groups of genes' expression values at a single time at said threshold value C;
ii. calculating an average number Cₐᵥₑ of said link counts Cₙ;
iii. calculating a largest number M of said Cₙ, wherein said M comprises the largest of said number of link counts Cₙ for a given said threshold value C for all said gene expression value groups;
iv. iteratively applying a relation Cₐᵥₑ=M/log(M) for different said threshold values C;
v. comparing data of said Cₐᵥₑ values versus M/log(M); and
vi. calculating a fitting to said compared data to output said scaling factor a, wherein said scaling factor a is the slope of said fitting;
c. comparing values of said scaling factor a for said at least one biological sample with other scaling factors a′ in a database from analyzed biological samples using said appropriately programmed computer; and
d. outputting a report using said appropriately programmed computer, wherein said report comprises estimates of said at least one biological sample for a degree of health.

2. The method of claim 1, wherein said at least one biological sample is selected from the group consisting of saliva, urine, other body fluids, synovial fluid, breast ductal fluid, blood and blood components, tissue, tumors, bone marrow, stem cells, induced pluripotent cells, cell lines, plant material, and other organic material.

3. The method of claim 1, wherein said gene expression reader comprises at least two gene probes.

4. The method of claim 1, wherein said number of link counts Cₙ comprises a number of link counts for each of N expression value groups, wherein each said expression value group comprises a sequence of gene expression values n₁, n₂, . . . n_{T}, at a threshold value C between said expression value group and said sequence of gene expression values n₁, n₂, . . . n_{T} for the other N−1 gene expression value groups.

5. The method of claim 1, wherein said scaling factor a is calculated by iteratively applying said Cₐᵥₑ=M/log(M) for different said threshold values C, using said appropriately programmed computer, and comparing Cₐᵥₑ values versus M/log(M) and calculating a linear fitting of said comparison to get said scaling factor a.

6. The method of claim 1, wherein said comparing values of said a further comprises comparing byproducts of said scaling factor a, comparing healthy samples against disease samples, or comparing an unknown sample with a database of values from samples with a known condition.

7. The method of claim 1, wherein said threshold value C is in a range between 0 and 1.

8. A system for diagnosing disease, comprising:
a. a gene expression reader for analyzing at least one biological sample, wherein said gene expression reader comprises a probe interfacing said at least one biological sample, wherein said probe comprises a fragment of nucleic acid having a specific sequence of bases that uniquely match a region of interest of a gene in a genome of said biological sample, wherein said probe interrogates a specific gene or a region within said specific gene of said biological sample, wherein said probe quantifies the expression level of said gene in said biological sample and outputting gene expression values of at least two genes;
b. a computer server for receiving from said gene expression reader said gene expression values and for managing and communicating patient information to a user; and
c. a computer program hosted on said computer server, wherein said computer program analyzes said gene expression values and outputs a report, wherein said report comprises estimates of said at least one biological sample for a degree of health, wherein said estimate comprises comparing a scaling factor a for said at least one biological sample with other scaling factors a′ in a database from previously analyzed biological samples, wherein said scaling factor a is calculated from said gene expression values using said computer program comprising:
i. counting a number of link counts Cₙ for groups of individual genes' expression values at a different times at a threshold value C or for groups of genes' expression values at a single time at said threshold value C;
ii. calculating an average number Cₐᵥₑ of said link counts Cₙ;
iii. calculating a largest number M of said Cₙ, wherein said M comprises the largest of said number of link counts Cₙ for a given said threshold value C for all said gene expression value groups;
iv. iteratively applying a relation Cₐᵥₑ=M/log(M) for different said threshold values C;
v. comparing said Cₐᵥₑ data values versus M/log(M) data; and
vi. applying a fitting to said compared data to output said scaling factor a, wherein said scaling factor a is the slope of said fitting.

9. The system of claim 8, wherein said at least one biological sample is selected from the group consisting of saliva, urine, other body fluids, synovial fluid, breast ductal fluid, blood and blood components, tissue, tumors, bone marrow, stem cells, induced pluripotent cells, cell lines, plant material, and organic material.

10. The system of claim 8, wherein said gene expression reader comprises at least two gene probes.

11. The system of claim 8, wherein said number of link counts Cₙ comprises a number of link counts for each of N expression value groups, wherein each said expression value group comprises a sequence of gene expression values n₁, n₂, . . . n_{T}, at a threshold value C between said expression value group and said sequence of gene expression values n₁, n₂, . . . n_{T} for the other N−1 gene expression value groups.

12. The system of claim 8, wherein said a scaling factor a is calculated by iteratively applying said Cₐᵥₑ=M/log(M) for different said threshold values C, using said appropriately programmed computer, and comparing Cₐᵥₑ values versus M/log(M) and calculating a linear fitting of said comparison to get said scaling factor a.

13. The system of claim 8, wherein said comparing values of said a further comprises comparing byproducts of said scaling factor a, comparing healthy samples against disease samples, or comparing an unknown sample with a database of values from samples with a known condition.

14. The system of claim 8, wherein said threshold value C is in a range between 0 and 1.

15. A lab-on-a-chip device, comprising:
a. a substrate for holding a biological sample receptacle, a gene expression analyzer and a microprocessor, wherein said at least one biological sample receptacle comprises a sample input to said gene expression analyzer, wherein said gene expression analyzer outputs gene expression values of at least two genes based on analyzed said at least one biological sample, wherein said microprocessor comprises a computer program for analyzing gene expressions in said at least one biological sample, wherein said computer program:
i. compiles said gene expression values;
ii. counts a number of link counts Cₙ for groups of individual genes' expression values at different times at a threshold value C or for groups of genes' expression values at a single time at said threshold value C;
iii. calculates an average number Cₐᵥₑ of said link counts Cₙ;
iv. calculates a largest number M of said Cₙ, wherein said M comprises the largest of said number of link counts Cₙ for a given said threshold value C for all said gene expression value groups;
i. iteratively applies a relation Cₐᵥₑ=M/log(M) for different said threshold values C;
ii. compares data of said Cₐᵥₑ values versus M/log(M) data;
iii. calculates a fitting to said compared data to output said scaling factor a, wherein said scaling factor a is the slope of said fitting;
iv. compares values of said scaling factor a for said at least one biological sample with other stored scaling factors a′ from analyzed biological samples; and
v. outputs a report, wherein said report comprises estimates of said at least one biological sample for a degree of health.

16. The device of claim 15, wherein said at least one biological sample is selected from the group consisting of saliva, urine, other body fluids, synovial fluid, breast ductal fluid, blood and blood components, tissue, tumors, bone marrow, stem cells, induced pluripotent cells, cell lines, plant material, and organic material.

17. The device of claim 15, wherein said gene expression reader comprises at least two gene probes.

18. The device of claim 15, wherein said number of link counts Cₙ comprises a number of link counts for each of N expression value groups, wherein each said expression value group comprises a sequence of gene expression values n₁, n₂, . . . n_{T}, at a threshold value C between said expression value group and said sequence of gene expression values n₁, n₂, . . . n_{T} for the other N−1 gene expression value groups.

19. The device of claim 15, wherein said a scaling factor a is calculated by iteratively applying said Cₐᵥₑ=M/log(M) for different said threshold values C, using said appropriately programmed computer, and comparing Cₐᵥₑ values versus M/log(M) and calculating a linear fitting said comparison to get said scaling factor a.

20. The device of claim 15, wherein said comparing values of said a further comprises comparing byproducts of said scaling factor a, comparing healthy samples against disease samples, or comparing an unknown sample with a database of values from samples with a known condition.

21. The device of claim 15, wherein said threshold value C is in a range between 0 and 1.

22. A method comprising:
a. obtaining a saliva sample of a subject;
b. measuring an expression value of a gene in the saliva sample by contacting the saliva sample with a fluid composition comprising a gene probe;
c. comparing by a computer system the expression level of the gene in the saliva sample with a reference database of gene expression values; and
d. determining a degree of health of the subject based on the comparing.
